(19) United States
(12) Patent Application Publication
(10) Pub. No.: US 2006/0052757 A1
(43) Pub. Date: Mar. 9, 2006

(54) Implantable medical device with analgesic or anesthetic

(75) Inventors: Frank J. Fischer JR., Bloomington, IN (US); Jessica Watts Miller, Terre Haute, IN (US); Marvin O. Andrews, Bloomington, IN (US)

Correspondence Address: BRINKS HOFER GILSON & LIONE/CHICAGO/COOK, PO BOX 10395, CHICAGO, IL, 60610, US

(73) Assignee: Vance Products Incorporated, d/b/a Cook Urological Incorporated, Spencer, IN (US)

(21) Appl. No.: 11/236,776

(22) Filed: Sep. 27, 2005

Related U.S. Application Data
(63) Continuation-in-part of application No. 10/410,587, filed on Apr. 8, 2003.
(63) Continuation-in-part of application No. 08/868,518, filed on Jun. 4, 1997, now Pat. No. 6,599,275.
(60) Provisional application No. 60/018,924, filed on Jun. 4, 1996.

(51) Int. Cl. A61M 25/00

(52) U.S. Cl. 604/265

(57) ABSTRACT

An implantable medical device such as a catheter with a controlled-release outer layer including a pharmacologically active ingredient for helping to relieve pain associated with the implantation of the device. The device includes a base material with an outer bioactive material layer including, for example, an analgesic or a local anesthetic.

### RELATED APPLICATIONS

The present patent document is a continuation-in-part of application Ser. No. 10/410,587, filed on Apr. 8, 2003, which is a continuation-in-part of application Ser. No. 08/868,518, filed on Jun. 4, 1997, now U.S. Pat. No. 6,599,275, and entitled “Implantable Medical Device”, which claims the benefit of provisional application Ser. No. 60/018,924, filed on Jun. 4, 1996. Each of these applications and patents is hereby incorporated by reference in its entirety, as though they were reproduced within this document.

This application is also related to two applications filed on the same day as the present application, a first application, Ser. No. ______, entitled IMPLANTABLE MEDICAL DEVICE WITH PARMACOLOGICALLY ACTIVE LAYER, and a second application, Ser. No. ______ IMPLANTABLE MEDICAL DEVICE WITH ANTI-NEOPLASTIC DRUG.

### FIELD OF THE INVENTION

This invention relates generally to medical devices and, particularly, to medical devices that are implantable either partly or completely into a human or veterinary patient.

### BACKGROUND OF THE INVENTION

It has become common to treat a variety of medical conditions by introducing an implantable medical device partly or completely into the esophagus, trachea, colon, biliary tract, urinary tract, vascular system or other location within a human or veterinary patient. For example, many treatments of the vascular system entail the introduction of a device such as a stent, a catheter, a balloon, a wire guide, a cannula, or the like. However, when such a device is introduced into and manipulated through the vascular system, the blood vessel walls can be disturbed or injured. Clot formation or thrombosis often results at the injured site, causing stenosis or occlusion of the blood vessel. Moreover, if the medical device is left within the patient for an extended period of time, a thrombus often forms on the device itself, again causing stenosis or occlusion. As a result, the patient is placed at risk of a variety of complications, including heart attack, pulmonary embolism, and stroke. Thus, the use of such a medical device can entail the risk of precisely the problems that its use was intended to ameliorate.

Another problem associated with implantable medical devices and, more particularly, to partly implanted medical devices such as catheters percutaneously introduced into the vascular system of a patient for long-term hemodialysis or drug infusion is the risk of infection. This risk is also present with hyperalimentation (intravenous feeding) catheters which are percutaneously introduced into the patient. The urinary tract is another system of the patient in which an urethral catheter such as a well-known Foley catheter is introduced into the patient's bladder via the urethra for the drainage of urine.

An attempt to reduce the risk of infection is to use a bioactive material and/or pharmacologically active ingredient such as an antibiotic in conjunction with the catheter. Various coatings including antibiotics have been utilized in the past; however, the antibiotic typically is dispersed or dissipated from the coating in a relatively short period of time. Although effective in short-term implantation, such coatings are typically ineffective for extended duration placement such as with hemodialysis, drug infusion, or urinary tract catheters, which can be implanted in the patient for two to three years at a time. What is needed is a better implant and a better way to coat the implant for a long term effect from an antiseptic or antimicrobial coating.

One proposal to reduce the risk of infection is directed to a partly implantable medical device such as an implantable catheter. The catheter includes an inner, elongated tube with an elongated outer sheath coaxially positioned around the inner tube. The size of the inner tube and outer sheath are selected to establish an intermediate space between the inner tube and outer sheath. An antibiotic drug or a mixture of antibiotic drugs is positioned or injected into the intermediate space. The material of the outer sheath is permeable to the antibiotic drug for diffusing the drug through the outer sheath at a given rate. The material of the inner tube can also be selected to be permeable to the drug for slowly diffusing the drug into the passage of the inner tube. However, when a mixture of drugs having different diffusion rates is positioned in the intermediate catheter space, the higher diffusion rate drug is quickly diffused through the inner tube and outer sheath without the benefit of the lower diffusion rate drug therewith for concomitantly combating the risk of infection. What is needed is a better way to control the time-rate of diffusion of a drug or drugs through the inner tube or the outer tube or both. What is also needed are better materials to be used as outer coatings that will constitute tubes.

### SUMMARY OF THE INVENTION

The foregoing problems are solved and a technical advance is achieved in an improvement to a medical device that is implantable either partly or completely into a human or veterinary patient. As previously suggested, the implantable medical device includes a pharmacologically active ingredient. The medical device is permeable to the pharmacologically active ingredient for diffusing the pharmacologically active ingredient therethrough.

When the pharmacologically active ingredient includes a mixture of ingredients, the bioactive or pharcologically active material can advantageously include one of the slower diffusion rate ingredients of the mixture, which is included in the base material of the selected member. This slower diffusion rate ingredient is then advantageously more readily accessible to the tissue surrounding the device for concomitant treatment with the other higher diffusion rate ingredient(s) of the mixture.

By way of example, the pharmacologically active ingredient advantageously and preferably includes a mixture of minocycline and rifampin, which is deposited in at least one layer on the surface of the implantable medical device. Minocycline has a lower diffusion rate than that of rifampin, and as a result, is included as the bioactive material in the device. The higher diffusion rate rifampin permeates through the surface layer on the elongated member and is diffused with the lower diffusion rate minocycline for treatment of tissue surrounding the catheter or other implantable medical device.

In a preferred embodiment of the invention, the base material of the implantable medical device is silicone having a durometer in a range of 30 to 90 on the Shore A Hardness Scale. Other materials may be used. Medical devices contemplated by the present application include, among other devices, a ureteral stent, a urethral catheter, a biliary stent, a pancreatic stent, a catheter for suprapubic drainage, a catheter for nephrostomy drainage, a catheter for nasal pancreatic drainage, and a nasal biliary drainage catheter

One embodiment of the invention is an implantable medical device meant for insertion in a patient. The medical device includes an attachment layer on at least a portion of the medical device and a controlled-release outer layer on at least a portion of the attachment layer, wherein the outer layer further comprises at least one analgesic compound, wherein a majority portion of a cross-section of the medical device is not changed upon insertion into the patient.

Another aspect of the invention is a medical device for insertion into a patient. The medical device includes an attachment layer on at least a portion of the medical device, and a controlled-release outer layer on at least a portion of the attachment layer, the outer layer further comprising an analgesic compound and an additional pharmacologically active compound, and wherein a cross-section of a majority portion of the medical device is not changed upon insertion into the patient.

Another aspect of the invention is a method of treating a patient. The method includes furnishing an implantable medical device with an antimicrobial outer layer, and placing the device into the patient, wherein a cross section of the implantable medical device is not changed upon insertion into the patient.

### BRIEF DESCRIPTION OF THE DRAWINGS

FIG. 1 depicts a cross-sectioned end view of a preferred illustrative embodiment of the implantable medical device of the present invention;

FIG. 2 depicts a cross-sectioned end view of another preferred embodiment of the implantable medical device of the present invention; and

FIG. 3 depicts a partial, sectioned side view of the implantable medical device of FIG. 2;

FIG. 4 depicts a plan view of a Foley catheter; and

FIGS. 5A and 5B depict cross-sectional views of a ureteral stent.

### DETAILED DESCRIPTION OF THE PRESENTLY PREFERRED EMBODIMENTS

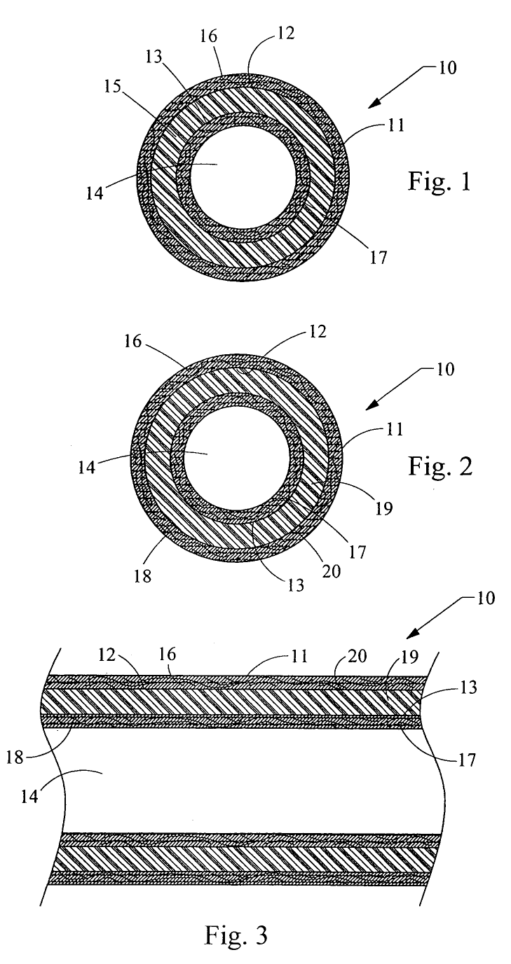

FIG. 1 depicts a cross-sectioned end view of a preferred illustrative embodiment of implantable medical device 10 such as a catheter having an outer, elongated member tube 11 with passage 12 extending longitudinally therein. Alternatively, outer elongated member tube can be simply a first layer 11 of material. Positioned concentrically and in passage 12 of outer elongated member tube 11 is inner elongated member tube 13 with passage 14 extending longitudinally therein. Again, alternatively, the inner elongated member tube can be simply a second layer 13 of material adjacent first layer 11. A tube or layer 15 of a pharmacologically active ingredient is positioned between and in communication with the outer and inner elongated member tubes or layers 11 and 13. The pharmacologically active ingredient is any drug, medicament, or agent for treating infections or other physiological afflictions encountered with the placement of such implantable medical devices.

Preferably, this pharmacologically active ingredient includes one or more drugs, agents, or medicaments for concomitantly minimizing or treating the infection or affliction. Preferably, this pharmacologically active ingredient would include a 50:50 mixture by weight of minocycline and rifampin. Minocycline has a lower diffusion rate than rifampin and, as a result, is also mixed in the base silicone material 16 of outer member tube 11 as or part of the bioactive material. The minocycline of the pharmacologically active ingredient is also included in the base silicone material 17 of inner elongated member tube 13. Minocycline 7% by weight in a powdered form is mixed with a powdered form of silicone and a solvent to form a liquid that is extruded into outer and inner member tubes 11 and 13. Inner elongated member tube 13 is positioned in passage 12 of outer elongated member tube 11. A desired length of the member tubes is cut to form the overall length of the catheter. One end of the catheter tubes is bonded together with a medical grade silicone adhesive.

By way of example, outer elongated member tube is approximately 0.125″ in diameter with a wall thickness of approximately 0.007″. Inner elongated member tube 13 has an inner diameter of approximately 0.062″ with a wall thickness of 0.007″. The pharmacologically active ingredient comprising a 50:50 mixture by weight of rifampin and minocycline is positioned, poured, or injected into the intermediate space between the inner and outer elongated member tubes 13 and 11. As a result, the wall or layer thickness of the pharmacologically active ingredient mixture is approximately 0.017″. The overall wall thickness of the catheter is approximately 0.031″.

Base silicone material 16 and 17 of the outer and inner members is preferably a silicone material having a durometer in a range of 30 to 90 on the Shore A Hardness Scale. Preferably, the minocycline and silicone mixture also has an overall durometer of 65 on the Shore A Hardness Scale. Base silicone material 16 and 17 is commercially available from the NU-SIL Corporation of Carpinteria, Calif.

Some embodiments are intended to provide a device in which at least two treatment materials reach the external or outer surfaces simultaneously. The implantable or partly implantable medical device includes a first elongated member or tube and a second elongated member or tube positioned adjacent to or within the first member or tube. A pharmacologically active ingredient is positioned between and in communication with the first and second elongated members. At least one of the first and second elongated members is permeable to the pharmacologically active ingredient for diffusing the pharmacologically active ingredient therethrough. A bioactive material preferably with a base material such as of at least one of the first and second elongated members is also provided, and the selected member(s) is permeable to the bioactive material for diffusing the bioactive material therefrom or therethrough. The second elongated member may be a coating or attachment layer on the first elongated member.

When the pharmacologically active ingredient material includes a mixture of ingredients, the pharmacologically active material can advantageously include one of the slower diffusion rate ingredients of the mixture, which is included in the base material of the selected member(s). This slower diffusion rate ingredient is then advantageously more readily accessible to the tissue surrounding the device for concomitant treatment with the other higher diffusion rate ingredient(s) of the mixture.

By way of example, the pharmacologically active ingredient advantageously and preferably includes a mixture of minocycline and rifampin, which is positioned between and in communication with the first and second elongated members of the implantable medical device. Minocycline has a lower diffusion rate than that of rifampin, and as a result, is included as the bioactive material in the base material in either one or both of the first and second elongated members. The higher diffusion rate rifampin permeates through the permeable base material of the elongated members and is diffused with the lower diffusion rate minocycline for concomitant treatment of tissue surrounding the outer surface of the catheter.

The thickness of the base material is advantageously selected to, in effect, slow down the diffusion of the higher diffusion rate ingredient so that the higher and lower diffusion rate ingredients are diffused from the medical device concomitantly for treatment of the tissues surrounding the device. An example of a combination drug in which different rates of diffusion may not be needed is gendine. Gendine is a mixture of gentian violet and chlorhexidine. Gentian violet may be any of several basic dyes that are derivatives of pararosaniline. The combination is useful with chlorhexidine as an antiseptic or antimicrobial agent.

FIG. 2 depicts a second preferred embodiment of implanted medical device 10 such as a catheter with outer elongated member tube 11 and inner elongated member tube 13 positioned in passage 12 of outer elongated member tube 11. An intermediate layer or tube 18 of a base material such as silicone is positioned between outer and inner elongated member tubes 11 and 13 and in the intermediate space therebetween. Base material 16, 17 and 19 of outer, inner and intermediate layer tubes 11, 13 and 18 is a medical grade silicone material from the NU-SIL Corporation. The 7% minocycline is included in base material 16 and 17 of outer and inner member tubes 11 and 13. Base silicone material 19 includes a bioactive material such as a 50:50 mixture by weight of rifampin and minocycline. This bioactive material mixture is 7% by weight of the base silicone material 19. The wall thickness of the inner, outer and intermediate layer tubes is as previously described to permit the higher diffusion rate of rifampin to mix and permeate through the layers and out the inner and inner surfaces of the catheter concomitantly. Alternatively, the catheter may include base material 19 with outer and inner layers 16, 17, each including a pharmacologically active ingredient. Interface layers 13, 18 may alternatively be interfaces between the catheter or stent base material 19 and layers 16, 17.

It is intended that the term pharmacologically active material or bioactive material includes any material that is molecularly interactive with the fluids, cells, proteins or tissues of an animal including humans to augment the diagnosis, treatment or prevention of any physiologic or pathologic condition. It is further intended that this term includes therapeutic and diagnostic agents such as, for example, drugs, vaccines, hormones, steroids, proteins, previously described agents, complexing agents, salts, chemical compounds, polymers, and the like.

In one embodiment, the base silicone material is a powdered material that is mixed with the bioactive material and/or the pharmacologically active ingredient in a well-known solvent. The mixture is then extruded at low temperatures with the solvent evaporating therefrom as the silicone material cures. This low temperature silicone is utilized so as not to evaporate the pharmacologically active ingredient and/or the bioactive material.

FIG. 3 depicts a partial, sectioned side view of medical device 10 of FIG. 2. Outer and inner elongated member tubes are likewise shown with intermediate tube or layer 18 positioned therebetween and in communication therewith. Passage 14 of the catheter is approximately one-half the outside diameter of catheter 10. As previously discussed, the overall wall thickness of catheter 10 is approximately 0.062″. The outside diameter of the catheter is again 0.025″. Intermediate tube or layer 18 is approximately two and one-half times the wall thickness of inner and outer elongated member tubes 13 and 11. Inner elongated member tube 13 is first extruded, with intermediate tube or layer 18 extruded thereover. Outer elongated member tube 11 is then extruded over the intermediate and inner elongated member tubes. As mentioned above, outer and inner layers 16, 17 with one or pharmacologically active ingredients may be interfaced to base material 19 with interface layers 13, 18.

With continued reference to FIGS. 1-3, implantable medical device 10 of the present invention comprises at least one bioactive material mixed with base material 16, 17 and/or 19 of outer, inner and/or intermediate layers 13, 11 and 18. For the purposes of the present invention, at least one bioactive material can also be posited on outer surface 20 of intermediate layer 18. The other surfaces of the outer, inner, and intermediate layers or the layers themselves can either contain no bioactive material or one or more different bioactive materials. In this manner, one or more bioactive materials or drugs may be delivered, for example, with a vascular stent or catheter, to the blood stream from the lumen surface of the stent, and a different treatment may be delivered on the vessel surface of the stent. A vast range of drugs, medicaments and materials may be employed as the bioactive material in one or more layers 16, 17 and 19, so long as the selected material can survive exposure to the placement or extrusion process or to a vacuum drawn during vapor deposition or plasma deposition. In other embodiments, other techniques may be used to apply the pharmacologically active ingredients, such as soaking or spraying.

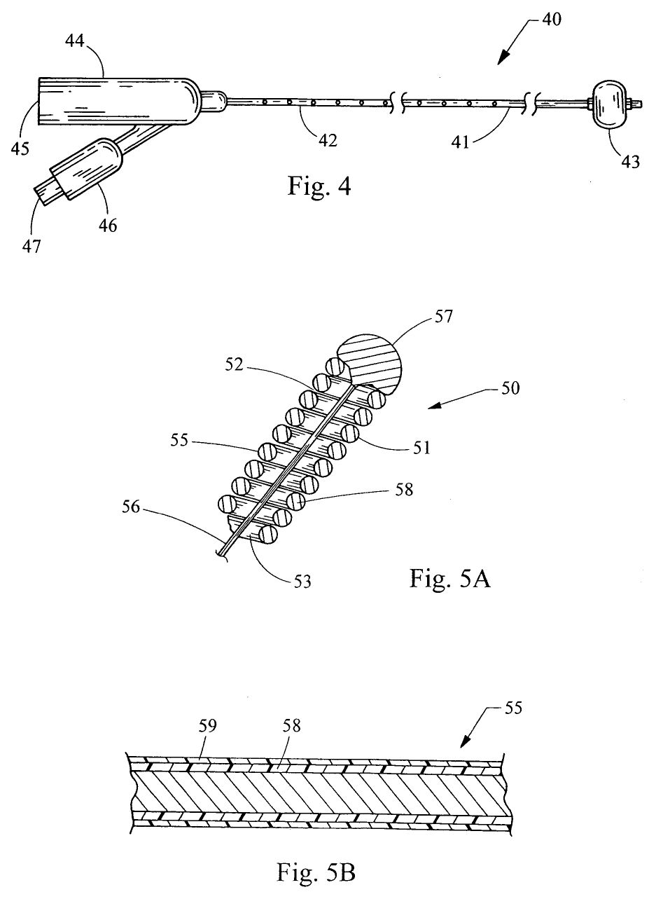

FIG. 4 depicts a Foley catheter 40. Foley catheter 40 includes a length 41 with a plurality of drainage holes 42 for draining urine from a urinary bladder of a patient. The Foley catheter has a constant cross section or diameter for most of its length, except for a retention balloon 43. Balloon 43 is placed into the patient's bladder and is then inflated using fitting 46 and inflation lumen 47. Urine is drained from the patient through outlet 45 and outlet fitting 44, which may be used to connect to a container, such as a drainage bag.

FIGS. 5A and 5B depict a ureteral stent. Ureteral stent 50 includes a length 51 of coiled wire 55 with spaces 52 between the coils so that urine can seep into central lumen 53. Stent 50 may have an internal rod 56 for securing end caps 57 to the stent. End caps 57 may also be secured to the end coils by soldering, welding, or brazing, or other joining technique. FIG. 5B depicts a cross section of wire 55 showing inner elongated member 55, intermediate layer 58 and outer layer 59.

Particularly useful in the practice of the present invention are materials which prevent or ameliorate abrupt closure and restenosis of blood vessels previously opened by stenting surgery or other procedures. Thrombolytics (which dissolve, break up or disperse thrombi) and antithrombogenics (which interfere with or prevent the formation of thrombi) are especially useful bioactive materials when the implantable medical device 10 is a vascular stent. Particularly preferred thrombolytics are urokinase, streptokinase, and the tissue plasminogen activators. Particularly preferred antithrombogenics are heparin, hirudin, and the antiplatelets.

Urokinase is a plasminogen activating enzyme typically obtained from human kidney cell cultures. Urokinase catalyzes the conversion of plasminogen into the fibrinolytic plasmin, which breaks down fibrin thrombi. Heparin is a mucopolysaccharide anticoagulant typically obtained from porcine intestinal mucosa or bovine lung. Heparin acts as a thrombin inhibitor by greatly enhancing the effects of the blood's endogenous antithrombin III. Thrombin, a potent enzyme in the coagulation cascade, is key in catalyzing the formation of fibrin. Therefore, by inhibiting thrombin, heparin inhibits the formation of fibrin thrombi. Alternatively, heparin may be covalently bound to the outer layer of implantable medical device 10. Thus, heparin would form the outermost layer of implantable medical device 10 and would not be readily degraded enzymatically, and would remain active as a thrombin inhibitor.

Of course, bioactive materials having other functions can also be successfully delivered by the device 10 of the present invention. For example, an antiproliferative agent such as methotrexate will inhibit over-proliferation of smooth muscle cells and thus inhibit restenosis of the dilated segment of the blood vessel. The antiproliferative is desirably supplied for this purpose over a period of about four to six months. Additionally, localized delivery of an antiproliferative agent is also useful for the treatment of a variety of malignant conditions characterized by highly vascular growth. In such cases, the device 10 of the present invention could be placed in the arterial supply of the tumor to provide a means of delivering a relatively high dose of the antiproliferative agent directly to the tumor.

A vasodilator such as a calcium channel blocker or a nitrate will suppress vasospasm, which is common following angioplasty procedures. Vasospasm occurs as a response to injury of a blood vessel, and the tendency toward vasospasm decreases as the vessel heals. Accordingly, the vasodilator is desirably supplied over a period of about two to three weeks. Of course, trauma from angioplasty is not the only vessel injury which can cause vasospasm, and the device 10 may be introduced into vessels other than the coronary arteries, such as the aorta, carotid arteries, renal arteries, iliac arteries or peripheral arteries for the prevention of vasospasm.

A variety of other bioactive materials are particularly suitable for use when the structure 12 is configured as something other than a coronary stent. For example, an anti-cancer chemotherapeutic agent can be delivered by the device 10 to a localized tumor. More particularly, the device 10 can be placed in an artery supplying blood to the tumor or elsewhere to deliver a relatively high and prolonged dose of the agent directly to the tumor, while limiting systemic exposure and toxicity. The agent may be a curative, a pre-operative debulker reducing the size of the tumor, or a palliative which eases the symptoms of the disease. It should be noted that the bioactive material in the present invention is delivered across the device 10, and not by passage from an outside source through any lumen defined in the device 10, such as through a catheter employed for conventional chemotherapy. The bioactive material of the present invention may, of course, be released from the device 10 into any lumen defined in the device, or to tissue in contact with the device and that the lumen may carry some other agent to be delivered through it. For example, tamoxifen citrate, Taxol® (paclitaxel) or derivatives thereof, Proscar® (finasteride) Hytrin® (terazosin hydrochloride) or Eulexin® (flutamide) may be applied to the tissue-exposed surface of the device for delivery to a tumor located, for example in breast tissue or the prostate. Docetaxel may also be used

Dopamine or a dopamine agonist such as bromocriptine mesylate or pergolide mesylate is useful for the treatment of neurological disorders such as Parkinson's disease. The device 10 could be placed in the vascular supply of the thalamic substantia nigra for this purpose, or elsewhere, localizing treatment in the thalamus.

A wide range of other bioactive materials can be delivered by the device 10. Accordingly, it is preferred that the bioactive material contained in or posited on the layer 18 includes at least one of heparin, covalent heparin, or another thrombin inhibitor, hirudin, hirulog, argatroban, D-phenylalanyl-L-poly-L-arginyl chloromethyl ketone, or another antithrombogenic agent, or mixtures thereof; urokinase, streptokinase, a tissue plasminogen activator, or another thrombolytic agent, or mixtures thereof; a fibrinolytic agent; a vasospasm inhibitor; a calcium channel blocker, a nitrate, nitric oxide, a nitric oxide promoter or another vasodilator; Hytrin® or other antihypertensive agents; an antimicrobial agent or antibiotic; aspirin, triclopidine, a glycoprotein IIb/IIIa inhibitor or another inhibitor of surface glycoprotein receptors, or another antiplatelet agent; colchicine or another antimitotic, or another microtubule inhibitor, dimethyl sulfoxide (DMSO), a retinoid or another antisecretory agent; cytochalasin or another actin inhibitor; or a remodeling inhibitor; deoxyribonucleic acid, an antisense nucleotide or another agent for molecular genetic intervention; methotrexate or another antimetabolite or antiproliferative agent; tamoxifen citrate, Taxol® or the derivatives thereof, or other anti-cancer chemotherapeutic agents; dexamethasone, dexamethasone sodium phosphate, dexamethasone acetate or another dexamethasone derivative, or another anti-inflammatory steroid or non-steroidal antiinflammatory agent; cyclosporin or another immunosuppressive agent; trapidal (a PDGF antagonist), angiopeptin (a growth hormone antagonist), angiogenin, a growth factor or an anti-growth factor antibody, or another growth factor antagonist; dopamine, bromocriptine mesylate, pergolide mesylate or another dopamine agonist; ⁶⁰Co (5.3 year half life), ¹⁹²Ir (73.8 days), ³²P (14.3 days), ¹¹¹In (68 hours), ⁹⁰Y (64 hours), ^{99m}Tc (6 hours) or another radiotherapeutic agent; iodine-containing compounds, barium-containing compounds, gold, tantalum, platinum, tungsten or another heavy metal functioning as a radiopaque agent; a peptide, a protein, an enzyme, an extracellular matrix component, a cellular component or another biologic agent; captopril, enalapril or another angiotensin converting enzyme (ACE) inhibitor; ascorbic acid, alpha tocopherol, superoxide dismutase, deferoxamine, a 21-aminosteroid (lasaroid) or another free radical scavenger, iron chelator or antioxidant; a ¹⁴C-, ³H-, ¹³¹I-, ³²P- or ³⁶S-radiolabelled form or other radiolabelled form of any of the foregoing; estrogen or another sex hormone; AZT or other antipolymerases; acyclovir, famciclovir, rimantadine hydrochloride, ganciclovir sodium, Norvir®, Crixivan®, or other antiviral agents; 5-aminolevulinic acid, meta-tetrahydroxyphenylchlorin, hexadecafluoro zinc phthalocyanine, tetramethyl hematoporphyrin, rhodamine 123 or other photodynamic therapy agents; an IgG2 Kappa antibody against Pseudomonas aeruginosa exotoxin A and reactive with A431 epidermoid carcinoma cells, monoclonal antibody against the noradrenergic enzyme dopamine beta-hydroxylase conjugated to saporin or other antibody targeted therapy agents; gene therapy agents; and enalapril and other prodrugs; Proscar®, Hytrin® or other agents for treating benign prostatic hyperplasia (BHP) or a mixture of any of these; and various forms of small intestine submucosa (SIS).

Other drugs that may be placed on the device to be implanted include analgesics and local anesthetics. A local anesthetic, such as lidocaine, relieves pain by inducing numbness, i.e., blocking the transmission of pain signals from the nerves to the brain. An analgesic, such as aspirin, helps to relieve pain by altering the perception of pain by the person without inducing anesthesia. Either a local anesthetic or an analgesic may be used with the medical device to be implanted in order to assist in patient comfort.

In addition to aspirin, other non-steroidal anti-inflammatory drugs that may be coated onto an insertable medical device include naproxen, choline, diflunisal, salsalate, fenoprofen, flurbiprofen, ketoprofen, ibuprofen, oxaprozin, diclofenac, indomethacin, sulindac, acetoaminophen, tolmetin, meloxicam, piroxicam, meclofenamate, mefanimic acid, nabumetone, etodelac, keterolac, celecoxib, valdecoxib and rofecoxib, mixtures thereof, and derivatives thereof. Other analgesics may include narcotics (opioids) and synthetic drugs with a narcotic effect, such as tramadol. Narcotics which may be coated onto medical devices include alfentanil, buprenorphine, carfentanil, codeine, codeinone, dihydrocodeine, dextropropoxyphene, endorphin, fentanyl, hydrocodone, hydromorphone, methadone, morphine, morphinone, oxycodone, oxymorphone, pethidine, remifantanil, sulfentanil, thebaine, and tramadol, mixtures thereof, and derivatives thereof.

Local anesthetics, with the same practical effect of pain relief, may also be coated onto the medical devices. Examples of local anesthetics include, but are not limited to, novacaine, bupivacaine, lidocaine, ropivacaine, and paracetamol. For purposes of this patent document, a local anesthetic, such as any of these examples, is considered to be the functional equivalent of an analgesic when coated onto a medical device for implantation into a patient, because both classical analgesics and anesthetics allow a patient to remain conscious without being aware of, or minimizing, pain. The term analgesic is thus intended to encompass a base compound and its derivatives, such as salts and esters that are physiologically effective to reduce the perception of pain at the treatment site. For purposes of the present patent document, a local anesthetic, and its derivates, salts and esters, is considered to be the functional equivalent of an analgesic.

Other pharmacologically active substances include additional drugs that are effective against urinary encrustation, in addition to heparin and other drugs listed above. These additional anti-encrustation drugs include triclosan, silver nitrate, ofloxacin, ciproflaxin, phosphorylcholine and trimethoprim. There are also additional drugs useful against microbes, including a penicillin, a cephalosporin, a carbepenem, a beta-lactam, an antibiotic, an aminoglycoside, a macrolide, a lincosamide, a glycopeptide, a tetracyline, a chloramphenicol, a quinolone, a fucidin, a sulfonamide, a trimethoprim, a rifamycin, an oxaline, a streptogramin, a lipopeptide, a ketolide, a polyene, an azole, and an echinocandin. Still other useful antimicrobial drugs with which an implantable medical device may be coated include alpha-terpineol, methylisothiazolone, cetylpyridinium chloride, chloroxyleneol, hexachlorophene, chlorhexidine and other cationic biguanides, methylene chloride, iodine and iodophores, triclosan, taurinamides, nitrofurantoin, methenamine, aldehydes, azylic acid, rifampycin, silver, benzyl peroxide, alcohols, and carboxylic acids and salts, and silver sulfadiazine. Also useful as antimicrobials are anthracyclines, such as doxorubicin or mitoxantrone, fluoropyrimidines such as 5-fluoroacil, and also podophylotoxins, such as etoposide. The salts and the derivatives of all of these are meant to be included as examples of antimicrobial drugs.

Another aspect of the invention is a method of preparation of the medical devices intended for complete or partial implantation in a patient. As mentioned above, preferred devices may be made from silicone or other polymers, such as urethane, and from derivatives of silicone and urethane, among other polymers. In addition to the technique described above for including a pharmacologically active ingredient within the base material itself, the desired pharmacologically active ingredient or ingredients may be applied in layers in order to control their release and rate of release. Certain of these methods are described in U.S. Pat. No. 5,759,708, and U.S. Pat. No. 5,958,430, which are hereby incorporated by reference in their entirety.

In one method of preparing an implantable medical device, the device is cleaned by being exposed to acetone and ethanol, and is then exposed to an air plasma for 20 minutes to remove organic residues. The substrate is then exposed sequentially to dilute KOH and HNO₃, rinsed, cleaned and dried. The substrate is then treated in a dilute alkenyl-silane solution, such as 1-(trichlorosilyl)-undec-10-ene. The terminal vinyl group of the alkenyl silane is converted to a sulfonic acid by exposure to SO₃ gas for one minute. The device is then cleaned again in deionized water and dried. Other methods of attaching functional groups to the surface of the substrate may be used. Desirable functional groups include carboxylates, sulfonates, phosphates, optionally substituted linear or cyclo alkyl, alkene, alkyne, aryl, alkylaryl, amine, hydroxyl, thiol, silyl, phosphoryl, cyano, metallocenyl, carbonyl, and polyphosphate.

After this interface or attachment layer is prepared, a further interface layer is also added. The desired drugs or pharmacologically active ingredients may then be added. In one embodiment, the interface is further prepared by repeatedly soaking the implantable device in a supersaturated solution of 5 mM CaCl₂, 1.5 mM KH₂PO₄, and 1.5 mM Na₂HPO₄. The solution is prepared by first adding the phosphates, and then slowly adding the calcium chloride to avoid precipitation. The devices are then soaked in this solution for about an hour or a lesser time in order to avoid precipitation. The process may be repeated as often as desired until the desired thickness is achieved. The resulting calcium phosphate films may be characterized by x-ray diffraction and scanning electron microscopy.

After the interface layer has achieved the desired thickness, the desired pharmacologically active ingredient or ingredients may be added by additional immersion cycles. A dilute solution of the desired ingredient or ingredients is prepared and the implantable devices are immersed for a period of time. The period of time depends on the rate of deposition of the drug. The devices are then rinsed and dried. If desired, additional cycles of immersion of the drug or drugs may be repeated. If the drug is soluble in the calcium chloride solution, the drug may be added to the calcium chloride solution and adsorbed onto the surface of the implantable device by simply repeating immersion cycles. If the drug is not soluble in the calcium chloride solution, a separate solution of the drug may be prepared and the implantable devices immersed separately.

The processing of the implantable devices may be completed by rinsing and drying the devices. Alternatively, one or more final layers may be applied, such as by spraying a top layer of the desired drug and a polymer. In one example, a dilute solution of the desired drug and a soluble polymer, such as polyvinyl chloride or polyvinyl alcohol, is sprayed over the implantable devices. The devices are then dried and packaged. In another embodiment, a hydrophilic coating is applied over the top sprayed layer, the hydrophilic coating applied by spraying or by immersion in a solution of a hydrophilic coating. The devices are then dried and packaged.

Medical devices according to the present invention are preferably fixed in size, having a constant cross-section, rather than being expandable in one or more dimensions. Examples are a double-pigtail ureteral stent or a urinary Foley catheter. These medical devices may have some variability in their inner diameter or outer diameter, i.e., in the sense that no device has perfect dimensional stability, and also in the sense that some parts of the device may be larger than other parts. When a ureteral stent is implanted into a ureter, or when a Foley catheter is implanted into a bladder and a urethra, there may be some compression of the walls of the stent or catheter, leading to a minor “change” in the inner diameter or outer diameter of the device. However, except for the balloon of the Foley catheter, these devices are not “radially expandable.” These devices are thus not similar to a vascular stent in which there is an intentional and desired change in the radial dimension so that the stent may be implanted and expanded to fulfill its intended purpose in a blood vessel. Implantable medical devices having a constant cross section include the urinary catheters and ureteral stents as described above, which have a majority portion with a constant cross section that does not change upon insertion. Medical devices, as the term is used herein, do not include vascular stents whose cross section and dimensions change abruptly upon implantation into a patient.

Permeation with a Pharmacologically Active Substance

U.S. Pat. No. 5,624,704, which is hereby incorporated by reference in its entirety, as though it were reproduced in this section word for word, gives several examples of non-metallic medical devices intended for implantation into a patient. One or more pharmacologically active substances, such as analgesics or anesthetics, may be impregnated into these devices by using the substances, a solvent and a penetrating substance. The solvent is preferably an organic solvent, and the penetrating agent is a substance that enables the pharmacologically active substance to permeate the base material or layers of the device intended for implantation.

The term “organic solvent” as used in the present invention means solvents that can be used to dissolve antimicrobial agents or pharmacologically active substances, the solvents including alcohols (i.e. methanol, ethanol), ketones (acetone, methylethylketone), ethers (tetrahydrofuran), aldehydes (formaldehyde), acetonitrile, acetic acid, methylene chloride and chloroform. The term “penetrating agent” as used in the present invention means an organic compound that can be used to promote penetration of the substance into the material of the medical device. Examples of these organic compounds are esters (i.e. ethyl acetate, propyl acetate, butyl acetate, amyl acetate, and combinations thereof), ketones (i.e. acetone and methylethylketone), methylene chloride and chloroform.

The term “alkalinizing agent” as used in the present invention means organic and inorganic bases including sodium hydroxide, potassium hydroxide, ammonia in water (27% ammonium hydroxide), diethylamine and triethylamine. The term “high ionic strength salts” as used in the present invention means salts exhibiting high ionic strength, such as sodium chloride, potassium chloride and ammonium acetate. These salts may act both as an alkalinizing agent and as a penetrating agent to enhance the receptivity of the medical implant material.

The term “bacterial and fungal organisms” as used in the present invention means all genuses and species of bacteria and fungi, including but not limited to all spherical, rod-shaped and spiral bacteria. Some examples of bacteria are stapylococci (i.e. Staphylococcus epidermidis, Staphylococcus aureus), Enterrococcus faecalis, Pseudomonas aeruginosa, Escherichia coli, other gram-positive bacteria and gram-negative bacilli. One example of a fungus is Candida albicans. Pharmacologically active substances may be effective to inhibit the growth of bacteria, fungi, or viruses, or may be cidal to bacteria, fungi or viruses.

The medical devices that are amenable to impregnation by pharmacologically active substances or combinations are generally comprised of a non-metallic material such as thermoplastic or polymeric materials. Examples of such materials are rubber, plastic, polyethylene, polyurethane, silicone, Gortex (polytetrafluoroethylene), Dacron® (polyethylene terephthalate), Teflon (polytetrafluoroethylene), latex, elastomers and Dacron sealed with gelatin, collagen or albumin.

Particular devices especially suited for application of the pharmacologically active substance combinations of this invention include urinary catheters, long term urinary devices, tissue bonding urinary devices, penile prostheses, vascular grafts, vascular catheter ports, wound drain tubes, hydrocephalus shunts, peritoneal catheters, pacemaker capsules, artificial urinary sphincters, small or temporary joint replacements, urinary dilators, heart valves and the like. Metallic devices coated with a non-metallic layer may also be impregnated as described herein.

One embodiment of the present invention is a method for impregnating a non-metallic medical implant with a pharmacologically active substance comprising the steps of forming a pharmacologically active substance of an effective concentration to inhibit the growth of bacterial, viral or fungal organisms by dissolving the substance in an organic solvent and adding a penetrating agent to the composition; and applying the substance to at least a portion of medical implant under conditions where the substance permeates the material of the medical implant.

In a preferred embodiment, the step of dissolving a pharmacologically active substance may also include the step of adding an alkalinizing agent to the composition in order to enhance the reactivity of the material of the medical implant. Further according to the preferred embodiment, the pharmacologically active substance is heated to a temperature between about 30° C. and 70° C. prior to applying the composition to the medical implant to increase the adherence of the pharmacologically active substance to the medical implant material. After the impregnated implant is removed from the solution of a pharmacologically active substance and allowed to dry, the impregnated implant is preferably rinsed with a liquid and milked to remove excess granular deposits and ensure uniform color of the impregnated implant. The pharmacologically active substance may be applied to the medical implant by dipping the implant into a solution of the dissolved substance for a period of between 15 and 120 minutes, and then removing the impregnated implant from the solution. Preferably, the implant is dipped in the composition for a period of approximately 60 minutes.

The method of the present invention preferably comprises a single step of applying a pharmacologically active substance to the surfaces of a medical implant. However, it is expected that several applications of the pharmacologically active substance, or other substances, can be applied to the surfaces of the implant without affecting the adherence of the pharmacologically active substance to the implant.

A preferred embodiment of the method for impregnating a catheter with a pharmacologically active substance comprises the steps of (1) forming a pharmacologically active substance of an effective concentration to inhibit the growth of bacterial, viral, and or organisms, such as staphylococci, other gram-positive bacteria, gram-negative bacilli and Candida, by (a) dissolving a pharmacologically active substance in an organic solvent, (b) adding a penetrating agent to the pharmacologically active substance and organic solvent composition, (c) adding an alkalinizing agent to the composition to improve the reactivity of the material of the medical implant; (2) heating the composition to a temperature of between about 30° C. and 70° C. to enhance the adherence of the pharmacologically active substance to the material of the medical device; (3) applying the pharmacologically active substance to the medical implant, preferably by dipping the implant in the composition for a period of about 60 minutes and under conditions where the pharmacologically active substance permeates the material of the medical device; (4) removing the impregnated medical implant from the pharmacologically active substance, and allowing it to dry; and (5) rinsing the impregnated medical implant with a liquid and milking the impregnated medical implant.

A further embodiment of the present invention is an implantable medical device comprising a medical implant comprising a non-metallic material, and a pharmacologically active substance, of an effective concentration to inhibit the growth of bacterial, viral or fungal organisms, coating the surface of the implant and impregnating the non-metallic material of the medical implant.

According to a preferred embodiment, the pharmacologically active composition comprises a mixture of an antimicrobial agent which may or may not be in solution, an organic solvent and a penetrating agent. The pharmacologically active substance composition may further comprise an alkalinizing agent. A preferred antimicrobial agent for use in pharmacologically active composition is a combination of minocycline and rifampin.

Basic Impregnation Method

450 mg of NaOH were dissolved in 45 ml of methanol while stirring until clear, yielding a concentration of 10 mg NaOH per ml of methanol. The dissolution was more rapidly achieved while stirring on a hot plate at a temperature of about 45° C. The final pH was about 12.1, taking into consideration that the pH in organic solvents may not be very reproducible. 4.5 g of minocycline were added in small aliquots over 1 hour to the above solution while stirring at a temperature of about 45° C. until clear. Then 9 g of rifampin were added in small aliquots over 15 minutes while stirring at a temperature of about 45° C. until clear. 255 ml of butyl acetate (pre warmed to 45° C.) were added in aliquots to the above solution while continuously stirring at 45° C. to keep the solution clear (antibiotics dissolve much more in methanol than in butyl acetate). Catheters (whole silicone catheters, polyurethane shafts and polyethylene shafts) were dipped in the solution, which contains 15 mg of minocycline and 30 mg of rifampin per ml of the 15:85 mixture of methanol:butyl acetate, for 1 hour at 45° C.

Catheters were removed from the antimicrobial solution and allowed to dry for at least 8 hours (preferably overnight). Catheters were then rinsed and gently milked under the water faucet to ensure uniform color, then allowed to dry for at least 2 hours before testing. It was noted that the uniform color of the catheters impregnated with the antimicrobial agent by the method of the present invention did not appreciably change by rinsing or even milking in water.

The impregnated catheters were then suspended in human urine for 7 days. The suspending urine was changed at day 3 and all catheters were suspended in urine from the same source. Table 1 summarizes the results of the zones of inhibition (Z.I.) produced by 18-fr silicone, 18-fr polyurethane and 16-fr polyethylene urinary catheters (all of these urinary catheters have a diameter of about 4 mm) at various intervals (D0: initially prior to suspension in urine; D1: one day after suspension; D7: seven days after suspension; ND: not done). A zone of inhibition of 10 mm or greater indicated antimicrobial efficacy.

TABLE 1

| | | Zone of Inhibition in mm | | |
|---|---|---|---|---|
| Catheter | Organism | D0 | D1 | D7 |
| 18-fr silicon | E. coli | 29 | 22 | 12 |
| 18-fr polyurethane | E. coli | 31 | 25 | 18 |
| 16-fr polyethylene | E. coli | ND | 8 | 7 |
| 18-fr silicon | P. aerug. | 22 | ND | 10 |
| 18-fr polyurethane | P. aerug. | 29 | ND | 12 |
| 16-fr polyethylene | P. aerug. | ND | ND | 5 |

Particularly preferred in the method for permeation is the use of a combination of methanol and butyl acetate, in a volume ratio of 15 parts methanol to 85 parts of butyl acetate. However, other ratios may be used, such as a 50:50 mixture by volume. Also useful, but not required, is the addition of 0-10 mg NaOH per ml of methanol. Later testing showed that polyurethane and silicone catheters were more easily permeated than polyethylene catheters, and that gas sterilization of impregnated catheters with ethylene oxide did not significantly affect the efficacy of at least antimicrobial compounds.

Another known method of coating the devices would be to first apply or absorb to the surface of the medical device a layer of tridodecylmethyl ammonium chloride (TDMAC) surfactant followed by an antibiotic coating layer. For example, a medical device having a polymeric surface, such as polyethylene, silastic elastomers, polytetrafluoroethylene or polyethylene terephthalate, can be soaked in a 5% by weight solution of TDMAC for 30 minutes at room temperature, air dried, and rinsed in water to remove excess TDMAC. Alternatively, TDMAC precoated catheters are commercially available. For example, central vascular catheters coated with TDMAC are available from Cook Critical Care, Bloomington, Ind. The device carrying the absorbed TDMAC surfactant coating can then be incubated in an antibiotic solution for up to one hour or so, allowed to dry, then washed in sterile water to remove unbound antibiotic and stored in a sterile package until ready for implantation. In general, the antibiotic solution is composed of a concentration of 0.01 mg/ml to 60 mg/ml of each antibiotic in an aqueous pH 7.4-7.6 buffered solution, sterile water, or methanol. According to one method, an antibiotic solution of 60 mg of minocycline and 30 mg of rifampin per ml of solution is applied to the TDMAC coated catheter.

It is to be understood, however, that the above-described implantable medical device is merely an illustrative embodiment of the principles of this invention, and that other devices and methods for using them may be devised by those skilled in the art, without departing from the spirit and scope of the invention. It is to be understood that the invention is directed to embodiments both comprising and consisting of the disclosed parts. It is contemplated that the entire device or only parts of the device can include the bioactive material and/or the pharmacologically active ingredient. Furthermore, different parts of the device can include different bioactive materials. It is also contemplated that different sides or regions of the same part of the device can include different bioactive materials or layers.

What is claimed is:

1. An implantable medical device, comprising:
a medical device for insertion into a patient;
an attachment layer on at least a portion of the medical device; and
a controlled-release outer layer on at least a portion of the attachment layer, wherein the controlled-release layer further comprises at least one analgesic compound.

2. The device of claim 1, wherein the analgesic compound is selected from the group consisting of aspirin and non-steroidal anti-inflammatory compounds.

3. The device of claim 1, wherein the controlled-release outer layer further comprises an antimicrobial compound selected from the group consisting of a penicillin, a cephalosporin, a carbepenem, a beta-lactam, an antibiotic, an aminoglycoside, a macrolide, a lincosamide, a glycopeptide, a tetracyline, a chloramphenicol, a quinolone, a fucidin, a sulfonamide, a trimethoprim, a rifamycin, an oxaline, a streptogramin, a lipopeptide, a ketolide, a polyene, an azole, and an echinocandin.

4. The device of claim 1, wherein the controlled-release outer layer further comprises an antimicrobial compound selected from the group consisting of alpha-terpineol, methylisothiazolone, cetylpyridinium chloride, chloroxyleneol, hexachlorophene, chlorhexidine and other cationic biguanides, methylene chloride, iodine and iodophores, triclosan, taurinamides, nitrofurantoin, methenamine, aldehydes, azylic acid, rifampycin, silver, benzyl peroxide, alcohols, and carboxylic acids and salts, silver sulfadiazine, an anthracycline, a fluoropyrimidine, a podophylotoxin, and an etoposide.

5. The device of claim 1, wherein the analgesic is selected from the group consisting of aspirin, naproxen, choline, diflunisal, salsalate, fenoprofen, flurbiprofen, ketoprofen, ibuprofen, oxaprozin, diclofenac, indomethacin, sulindac, acetoaminophen, tolmetin, meloxicam, piroxicam, meclofenamate, mefanimic acid, nabumetone, etodelac, keterolac, celecoxib, valdecoxib and rofecoxib, mixtures thereof, and derivatives thereof.

6. The device of claim 1, wherein the analgesic is selected from the group consisting of opioids, synthetic drugs with narcotic properties, and local anesthetics to include at least paracetamol, bupivacaine, ropivacaine, lidocaine, and novocaine.

7. The device of claim 1, wherein the medical device is selected from the group consisting of a ureteral stent, a urethral catheter, a biliary stent, a pancreatic stent, a catheter for suprapubic drainage, a catheter for nephrostomy drainage, a catheter for nasal pancreatic drainage, and a nasal biliary drainage catheter.

8. The device of claim 7, wherein walls of the medical device comprise a metallic coil or a polymer.

9. The device of claim 1, wherein the compound is released in measurable amounts over a period of at least one week.

10. The device of claim 1, wherein the controlled-release outer layer further comprises an anti-encrustation compound selected from the group consisting of heparin, covalent heparin, dexamethazone, dexamethasone sodium phosphate, dexamethasone acetate and another dexamethasone derivative.

11. The device of claim 1, wherein the controlled-release outer layer further comprises an anti-encrustation compound, an antispasmodic compound, or an antiseptic compound.

12. The device of claim 1, wherein the controlled-release outer layer further comprises an anti-encrustation compound selected from the group consisting of triclosan, silver nitrate, ofloxacin, ciproflaxin, phosphorylcholine and triemethoprim.

13. An implantable medical device, comprising:
a medical device for insertion into a patient;
an attachment layer on at least a portion of the medical device; and
a controlled-release outer layer on at least a portion of the attachment layer, wherein the controlled-release outer layer comprises an analgesic and an additional compound.

14. The implantable device of claim 13, wherein the additional compound is heparin, covalent heparin, dexamethazone, dexamethasone sodium phosphate, dexamethasone acetate or another dexamethasone derivative.

15. The implantable device of claim 13, wherein the analgesic is selected from the group consisting of non-steroidal anti-inflammatory drugs and aspirin.

16. The implantable device of claim 13, wherein the additional compound is gendine.

17. The implantable device of claim 13, wherein the additional compound is paclitaxel.

18. The implantable device of claim 13, wherein the additional compound is an anesthetic.

19. An implantable medical device, comprising:
a medical device for insertion into a patient;
an attachment layer on at least a portion of the medical device; and
a controlled-release outer layer on at least a portion of the attachment layer, wherein the controlled-release outer layer comprises an anesthetic.

20. The implantable medical device according to claim 19, wherein the anesthetic is selected from the group consisting of paracetamol, bupivacaine, ropivacaine, lidocaine, and novocaine

21. A method of treating a patient, the method comprising:
furnishing an implantable medical device with a controlled-release outer layer including an analgesic or an anesthetic; and
placing the device into the patient.

22. The method of claim 21, wherein the implantable medical device further comprises an analgesic.

23. The method of claim 21, wherein the implantable medical device further comprises a narcotic selected from the group consisting of alfentanil, buprenorphine, carfentanil, codeine, codeinone, dextropropoxyphene, dihydrocodeine, endorphin, fentanyl, hydrocodone, hydromorphone, methadone, morphine, morphinone, oxycodone, oxymorphone, pethidine, remifantanil, sulfentanil, thebaine, and tramadol, mixtures thereof, and derivatives thereof.
